(19) United States
(12) Patent Application Publication
(10) Pub. No.: US 2026/0105602 A1
(43) Pub. Date: Apr. 16, 2026

(54) MEDICAL IMAGE DIAGNOSIS SUPPORT APPARATUS, DIAGNOSIS SUPPORT APPARATUS, DIAGNOSIS SUPPORT METHOD, AND STORAGE MEDIUM

(71) Applicant: CANON MEDICAL SYSTEMS CORPORATION, Otawara-shi (JP)

(72) Inventors: Sho SASAKI, Ota (JP); Minoru NAKATSUGAWA, Yokohama (JP); Kosuke ARITA, Kawasaki (JP); Asateru KIMURA, Yokohama (JP); Takuo NEGISHI, Nasushiobara (JP)

(73) Assignee: CANON MEDICAL SYSTEMS CORPORATION, Otawara-shi (JP)

(21) Appl. No.: 19/352,773

(22) Filed: Oct. 8, 2025

(30) Foreign Application Priority Data
Oct. 11, 2024 (JP) 2024-179254

(51) Int. Cl. G06T 7/00; A61B 6/00; G16H 50/30

(52) U.S. Cl. CPC G06T 7/0012; G16H 50/30; A61B 6/5217; G06T 2207/30004

(57) ABSTRACT

A medical image diagnosis support apparatus of embodiments includes processing circuitry. The processing circuitry is configured to analyze a medical image of a subject captured by a medical imaging device to acquire information regarding a disease risk of the subject, calculate a credibility of the acquired information regarding the disease risk, determine additional image information desired to be acquired in addition to the acquired information regarding the disease risk and a destination of request for the additional image information based on the calculated credibility, and output request information for requesting provision of the determined additional image information from the destination of request.

### CROSS-REFERENCE TO RELATED APPLICATION

The present application claims priority based on Japanese Patent Application No. 2024-179254 filed Oct. 11, 2024, the content of which is incorporated herein by reference.

### FIELD

Embodiments disclosed in this specification and drawings relate to a medical image diagnosis support apparatus, a diagnosis support apparatus, a diagnosis support method, and a storage medium.

### BACKGROUND

A clinical decision support system (CDS) is known as a support function for doctors and other medical professionals to correctly ascertain a patient's condition and make an appropriate diagnosis. The CDS has a function of collecting and processing patient information and transmitting the same to medical professionals. In this sense, the CDS can be said to be a medium for establishing communication (information transmission) within a medical system. A medium is a means of intermediary that suppresses variations in semantic interpretation and conveys the meaning and content of information as accurately as possible.

As a medium, the CDS requires both a patient and a medical professional to “confirm” that “correct patient information is handled.” This can be explained by paradigmaticity (interactive understanding of the meaning and content of information) that is the characteristic of media. However, in emergency situations where a patient is unconscious, medical professionals may be the only ones collecting patient information, potentially resulting in insufficient confirmation from the patient. Furthermore, in the midst of a hectic situation, medical professionals may overlook certain details. For example, information that requires confirmation by the patient's family (such as allergies) should also be managed by the patient's family. Furthermore, the relationship between a patient and a medical professional that has been built through regular visits to the hospital may lead to bias in transmission and receipt of information. For example, in the case of chronic illnesses, it may be difficult to notice gradual changes in the patient's condition over time, and oversights about whether correct patient information is always being handled may go unnoticed for long periods of time.

In other words, conventional CDSs only store patient information obtained by determination of medical professionals, and the accuracy of that information is often not confirmed by the patient. Furthermore, the accuracy of information confirmed by medical professionals is only partial (human perception is limited). For this reason, a CDS as a medium is necessary to reduce the risk of misdiagnosis.

### BRIEF DESCRIPTION OF THE DRAWINGS

FIG. 1 is a diagram showing an example of a configuration of a medical information processing system S including a diagnosis support apparatus 1 according to a first embodiment.

FIG. 2 is a diagram showing an example of patient information according to the first embodiment.

FIG. 3 is a diagram showing examples of conditions used in calculating a credibility score according to the first embodiment.

FIG. 4 is a flowchart showing an example of medical support processing by the diagnosis support apparatus 1 according to the first embodiment.

FIG. 5 is a diagram showing an example of request information (for doctors and patients) according to the first embodiment.

FIG. 6A is a diagram showing an example of request information (for doctors) according to the first embodiment.

FIG. 6B is a diagram showing an example of request information (for patients) according to the first embodiment.

FIG. 7A is a diagram showing an example of input/output of an LLM according to a second embodiment.

FIG. 7B is a diagram showing another example of input/output of the LLM according to the second embodiment.

FIG. 7C is a diagram showing another example of input/output of the LLM according to the second embodiment.

FIG. 8 is a diagram showing an example of patient information according to a third embodiment.

### DETAILED DESCRIPTION

Hereinafter, a medical image diagnosis support apparatus, a diagnosis support apparatus, diagnosis support method, and a storage medium according to embodiments will be described with reference to the drawings. The diagnosis support apparatus according to embodiments provides a means for accurately, completely, and continuously managing subject information under the supervision of both a subject and a medical professional. That is, this diagnosis support apparatus provides functions of a clinical decision support system (hereinafter also referred to as “CDS-M”) as a medium. To improve the credibility of subject information, the CDS-M determines and presents a destination (subject, medical professional, or the like) of request for additional information that is desired to be newly collected and the content of the additional information. The CDS-M then updates the subject information on the basis of the collected additional information. The additional information may be additional image information. Medical professionals include, for example, doctors, nurses, technicians, etc. Subjects include, for example, patients. The following description takes, as an example, a case in which a medical professional is a doctor and a subject is a patient.

The medical image diagnosis support apparatus of embodiments includes processing circuitry. The processing circuitry is configured to analyze a medical image of a subject captured by a medical imaging apparatus to acquire information regarding a disease risk of the subject, calculate a credibility of the acquired information regarding the disease risk, determine additional image information desired to be acquired in addition to the acquired information regarding the disease risk and a destination of request for the additional image information based on the calculated credibility, and output request information for requesting provision of the determined additional image information from the destination of request.

### First Embodiment

### [System Configuration]

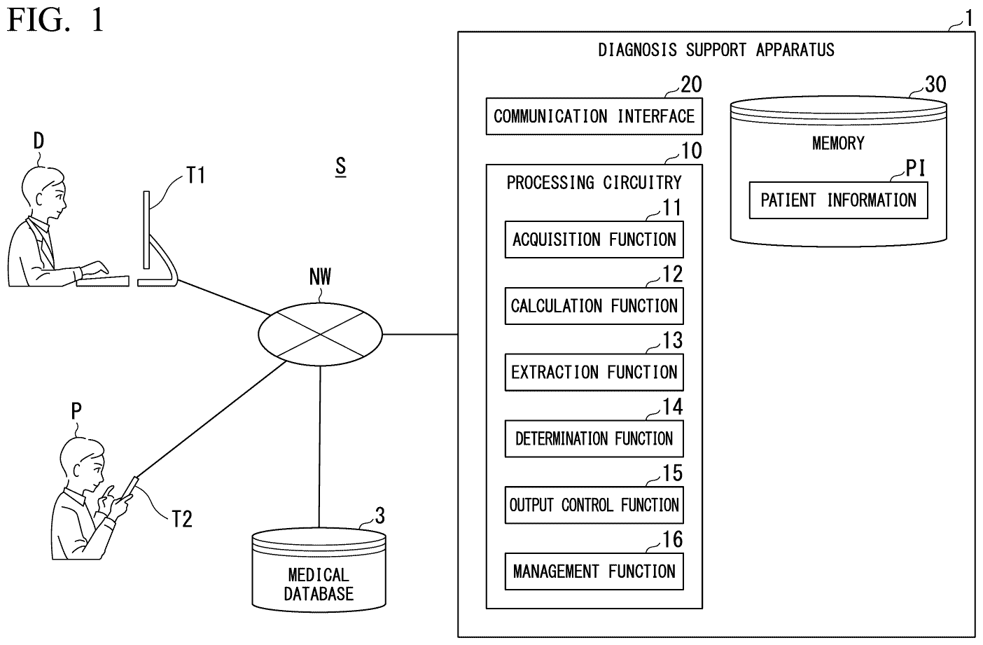

FIG. 1 is a diagram showing an example of a configuration of a medical information processing system S including a diagnosis support apparatus 1 according to a first embodiment. The medical information processing system S includes, for example, the diagnosis support apparatus 1 which manages information on target patients (hereinafter referred to as “patient information”), a medical database 3 which stores information that is the source of the patient information, at least one doctor terminal device T1, and at least one patient terminal device T2. These devices and equipment are connected to each other such that they can transmit and receive data via, for example, a communication network NW. The communication network NW includes telephone communication networks, optical fiber communication networks, cable communication networks, satellite communication networks, and the like in addition to wireless/wired local area networks (LAN) such as a hospital backbone LAN and the Internet.

### [Patient Information]

In the present embodiment, patient information refers to various types of information used in patient's medical care, among all information regarding the patient. Patient information is a collection of attributes that describe an individual patient in any clinical setting. Attributes include, for example, physical measurement values such as height and weight, vital signs such as blood pressure and pulse rate, blood test results, the presence or absence of disease, and other information obtained from patient's responses to questions on a medical questionnaire (lifestyle habits, motor function status, cognitive function status, skin and hair condition, personality traits, stress and sleep status, dietary habits, etc.). Patient information may be time-series data. Patient information may also be a time-series model. A time-series model is data in the format of “patient information 20240501 for patient A, patient information 20240504 for patient A, . . . ,” for example. If a time-series model can be logically constructed correctly (by applying a causal inference technique, or the like), it is possible to solve the problem of conjunction (realizing sequential communication connections) in the CDS-M.

For patient information, a “credibility score” is calculated for each attribute. The credibility score is an index value that indicates the certainty of the attribute (the certainty of patient information). For example, the credibility score is set such that it increases as the certainty of the attribute increases and decreases as the certainty of the attribute decreases. The diagnosis support apparatus 1 basically performs processing to improve this credibility score. The diagnosis support apparatus 1 presents patient information to a doctor and/or a patient. At that time, for attributes with low credibility scores, the diagnosis support apparatus 1 requests provision of additional information from the doctor and/or the patient. In response, the doctor and/or the patient provides the additional information, and the diagnosis support apparatus 1 updates the patient information on the basis of the obtained additional information. Patient information is an example of “subject information.” A credibility score is an example of “credibility.” Additional information here may also be additional image information.

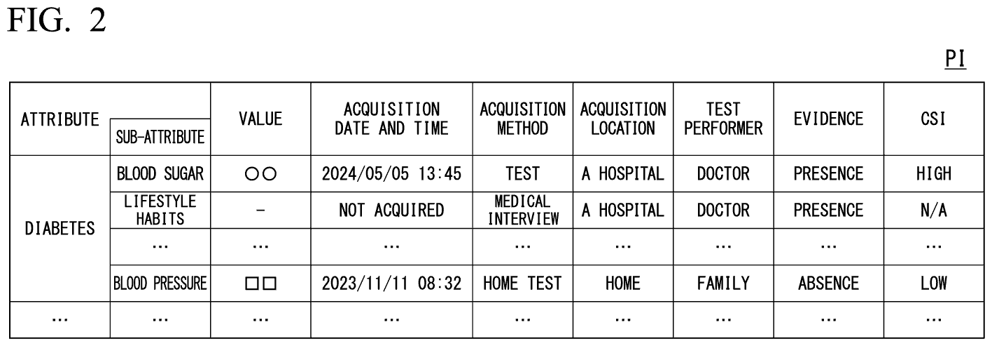

FIG. 2 is a diagram showing an example of patient information according to the first embodiment. As shown in FIG. 2, patient information includes at least one attribute. Each attribute is associated with at least one sub-attribute. Each sub-attribute is associated with at least one data item and a credibility score. For example, the attribute “diabetes” is associated with sub-attributes such as “blood sugar” and “lifestyle habits.” Further, each sub-attribute is associated with data items such as value, acquisition date and time, acquisition method, acquisition location, test performer, and evidence. Further, each sub-attribute is associated with a credibility score. Sub-attributes may not be set, and a data item may be associated with a credibility score for each attribute.

### [Diagnosis Support Apparatus]

Referring back to FIG. 1, the diagnosis support apparatus 1 controls the overall operation of the medical information processing system S and manages patient information. The diagnosis support apparatus 1 is an example of a “diagnosis support apparatus” or a “medical image diagnosis support apparatus.” The diagnosis support apparatus 1 may be, for example, a workstation, a server, or the like. The diagnosis support apparatus 1 includes, for example, processing circuitry 10, a communication interface 20, and a memory 30. The communication interface 20 communicates with external devices such as the medical database 3, the doctor terminal device T1, and the patient terminal device T2 via the communication network NW. The communication interface 20 includes, for example, a communication interface such as a network interface card (NIC).

The processing circuitry 10 controls the overall operation of the diagnosis support apparatus 1. The processing circuitry 10 includes, for example, an acquisition function 11, a calculation function 12, an extraction function 13, a determination function 14, an output control function 15, and a management function 16. The processing circuitry 10 realizes these functions by a hardware processor (computer) executing a program stored in the memory 30 (storage circuit), for example.

A hardware processor refers to circuitry such as a central processing unit (CPU), a graphics processing unit (GPU), an application specific integrated circuit (ASIC), or a programmable logic device (e.g., simple programmable logic device (SPLD), complex programmable logic device (CPLD), or field programmable gate array (FPGA)). Instead of storing the program in the memory 30, the hardware processor may be configured such that the program is directly embedded in the circuitry. In this case, the hardware processor realizes the functions by reading and executing the program embedded in the circuitry.

The aforementioned program may be stored in the memory 30 in advance, or may be stored on a non-transitory storage medium such as a DVD or a CD-ROM and installed into the memory 30 from the non-transitory storage medium by inserting the non-transitory storage medium into a drive device (not shown) of the diagnosis support apparatus 1. The hardware processor is not limited to being configured as a single circuit and may be configured as a single hardware processor combining a plurality of independent circuits to realize each function. Furthermore, a plurality of components may be integrated into a single hardware processor to realize each function.

The acquisition function 11 acquires various types of information (“information regarding a disease risk of a subject”) from an external device via the communications network NW. The acquisition function 11 acquires, for example, information that is a source of patient information (hereinafter also referred to as “source patient information”) from the medical database 3. Source patient information includes, for example, various types of patient test data (measurement values, images, etc.), electronic medical records, and response data to medical questionnaires (questionnaires). Alternatively, the acquisition function 11 may acquire test information from various other medical apparatus. For example, the acquisition function 11 may analyze medical images of a subject captured using any of medical imaging devices corresponding to various modalities, such as X-ray, CT, or MRI, to acquire information regarding a disease risk of the subject. The acquisition function 11 is an example of an “acquisition unit.”

The calculation function 12 calculates a credibility score of information (CSI) for the information acquired by the acquisition function 11. The calculation function 12 calculates the CSI, for example, from the perspective of the 5Ws (who, what, where, when, and why). The calculation function 12 calculates the CSI, for example, on the basis of the following expression (1).

$$CSI = {f}_{w h o} + {f}_{w h a t} + {f}_{w h e r e} + {f}_{when} + {f}_{w h y} \qquad ( 1 )$$

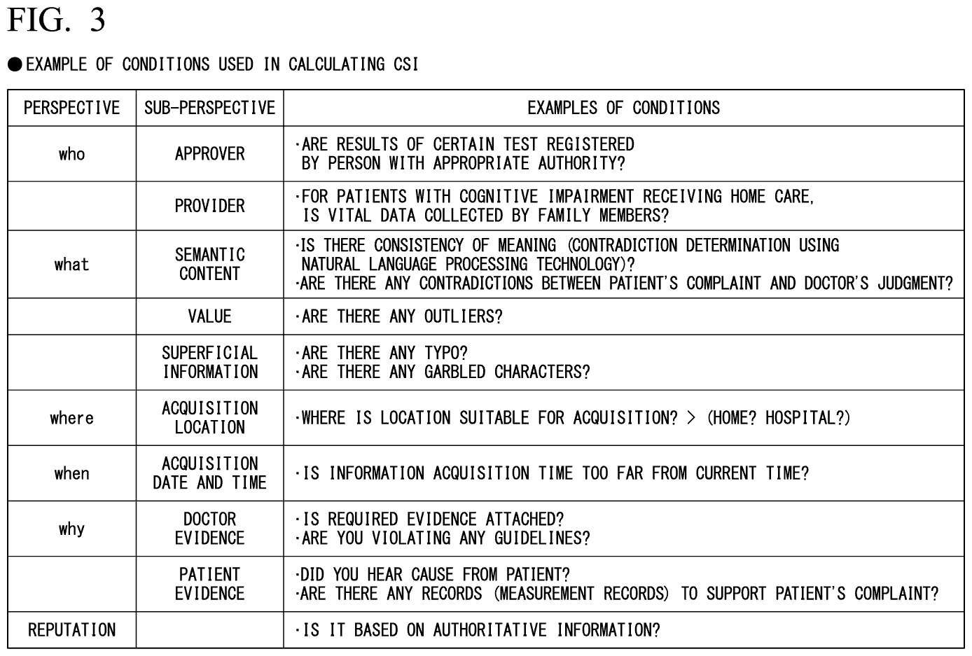

In the above expression (1), f is a determination function. The determination function f outputs “0” if a condition is satisfied and “−1” if the condition is not satisfied, for example. The determination function f for each perspective is defined based on the conditions shown in FIG. 3. For example, f_{who}, which is the determination function for the “who” perspective, has “approver” set as a sub-perspective, and a condition therefor is set as “Are the results of a certain test registered by a person with appropriate authority?.” f_{who} outputs “0” if patient information for which CSI is to be calculated satisfies this condition, and outputs “−1” if the patient information does not satisfy the condition. For example, in the example of patient information shown in FIG. 2, the data in the data item “test performer” is compared with reference data set in advance according to the type of attribute to determine whether the condition is satisfied.

Furthermore, for example, f_{when}, which is the determination function for the “when” perspective has “acquisition date and time” set as a sub-perspective, and a condition therefor is set as “Is the information acquisition time too far from the current time?.” f_{when} outputs “0” if the patient information for which CSI is to be calculated satisfies this condition, and outputs “−1” if the patient information does not satisfy the condition. For example, in the patient information example shown in FIG. 2, the data of the data item “acquisition date and time” is compared with the current time to determine whether the condition is satisfied.

For example, if the CSI calculated using the above expression (1) is less than 0, the calculation function 12 determines that the CSI is “low (Low CSI),” and if the CSI is 0, the calculation function 12 determines that the CSI is “high (High CSI),” and registers the determination result in the patient information. The calculation function 12 may also register the CSI value calculated using the above expression (1) in the patient information, for example.

It is not necessary to use all of the 5 W perspectives in expression (1) above, and some (at least one) of the 5 W perspectives may be used. Each perspective (e.g., “why”) may be defined separately as a doctor's perspective and a patient's perspective. A “reputation” perspective, which is different from the 5 W perspective, may also be set. A determination function f for this reputation perspective may be defined to perform determination processing based on conditions such as “whether it is based on authoritative information” and “whether there is a second opinion.”

The calculation function 12 is an example of a “calculation unit.” The SCI is an example of “credibility.” That is, the calculation function 12 calculates a CSI for information regarding a disease risk

Referring back to FIG. 1, the extraction function 13 extracts a predetermined number of pieces of patient information with low CSI when the patient information is sorted in order of size (degree, level) of CSI. For example, the extraction function 13 sorts attributes that have been determined to have “low CSI” in order of size of CSI, and extracts a predetermined number of attributes (for example, the three worst) in ascending order of CSI. The extraction function 13 is an example of an “extraction unit.”

The determination function 14 determines, for a predetermined number of pieces of patient information extracted by the extraction function 13, additional information that is desired to be acquired in addition to the information acquired by the acquisition function 11, and a destination of request for this additional information (depending on the CSI calculated by the calculation function 12). For example, the determination function 14 determines, as additional information, information for which there is no data in the attributes included in the patient information. For example, if the determination function f_{when} for the “when” perspective is “−1,” the determination function 14 determines information from the “when” perspective as additional information. Furthermore, if the CSI for the attribute “name of illness (e.g., cancer)” is low, the determination function 14 determines the doctor, rather than the patient, as the destination of request. Furthermore, if the SCI for an attribute that is information that is not supposed to be disclosed to the patient (e.g., in-hospital approval history) is low, the determination function 14 determines the doctor, rather than the patient, as the destination of request. The destination of request may include the patient's cohabitant or caregiver (family member, etc.) instead of the patient. The determination function 14 is an example of a “determination unit.” Based on the CSI, the determination function 14 determines additional information that is desired to be acquired in addition to the information regarding the disease risk and the destination of request for the additional information.

The output control function 15 outputs request information for requesting provision of the additional information from the destination of request. The output control function 15 outputs information to a terminal device (such as the doctor terminal device T1 and/or the patient terminal device T2) to request provision of the additional information from the destination of request (such as the doctor and/or the patient). This information includes information (attributes) regarding a disease risk, CSI, and character string information requesting provision of additional information from the destination of request. For example, if the determination function 14 determines information related to “when” perspective as additional information and determines the patient as that destination of request, the output control function 15 outputs information for displaying information containing a message, “Please tell me when you fell” to the patient terminal device T2. The output control function 15 may output request information in the form of a text message or speech (mechanical voice) instead of (or in addition to) information. The output control function 15 is an example of an “output control unit.”

The management function 16 manages patient information PI stored in the memory 30. The management function 16 associates CSI calculated by the calculation function 12 with attributes and stores the same in the memory 30 as patient information PI. In addition, the management function 16 also adds and deletes attributes included in patient information. For example, the management function 16 adds and deletes attributes and sub-attributes using information expressed in tree/graph formats, such as medical ontology and disease concept networks. Furthermore, if the electronic medical record created by a doctor contains a statement that “diabetes is suspected” (a statement of the disease name), the management function 16 adds an attribute (sub-attribute) related to “diabetes.” Furthermore, if the electronic medical record created by a doctor contains a statement that “the patient is experiencing convulsions” (a statement of symptoms), the management function 16 adds underlying diseases (cerebrovascular disease, metabolic disorder, etc.) and details that require interviewing (age of onset, medical history, family history, drug administration history, etc.) as attributes (sub-attributes). Alternatively, if the patient writes “I might have a fracture” (including the name of the disease) on a medical questionnaire, the management function 16 adds an attribute (sub-attribute) related to “fracture.” Furthermore, if the patient writes “My leg hurts” (including the symptom) on the medical questionnaire, the management function 16 adds underlying diseases (fracture, strain, etc.) and details that requires interviewing (the time of onset, etc.) as attributes (sub-attributes). Additionally, the management function 16 may update the attributes of patient information and the value of each data item on the basis of information in the hospital system, such as information obtained through medical interviews and input to the doctor terminal device T1. The management function 16 may also add attributes from guidelines or standard protocols (such as emergency situations).

The management function 16 is an example of a “management unit.” That is, the management function 16 stores information regarding disease risk and patient information based on CSI in a storage device.

The memory 30 is realized, for example, by a semiconductor memory element such as a random access memory (RAM), a flash memory, a hard disk, or an optical disk. These non-transitory storage media may also be realized by other storage devices connected via the communications network NW, such as a network attached storage (NAS) and an external storage server device. The memory 30 may also include other non-transitory storage media such as a read only memory (ROM) and a register. The memory 30 stores, for example, patient information PI. In addition, the memory 30 stores programs, parameter data, and other data used by the processing circuitry 10. The memory 30 is an example of a “storage device.”

### [Medical Database]

The medical database 3 is a storage device that stores source patient information, which is the source of patient information. For example, the medical database 3 stores various types of patient test data (measurement values, images, etc.), electronic medical records, and response data to medical questionnaires. The medical database 3 is realized, for example, using semiconductor memory devices such as a RAM and a flash memory, hard disks, or optical discs.

### [Doctor Terminal Device]

The doctor terminal device T1 is a device for referring to various types of information provided by the diagnosis support apparatus 1. The doctor terminal device T1 is operated, for example, by a doctor D. The doctor terminal device T1 is, for example, a personal computer or a mobile device such as a tablet or a smartphone. The doctor terminal device T1 includes, for example, a communication function for data communication with other devices, an input interface function for accepting various instructions from the doctor D, and a display function for displaying various types of information.

### [Patient Terminal Device]

The patient terminal device T2 is a device for referring to various types of information provided by the diagnosis support apparatus 1. The patient terminal device T2 is operated, for example, by a patient P. The patient terminal device T2 may be, for example, a personal computer or a mobile device such as a tablet or a smartphone. The patient terminal device T2 includes, for example, a communication function for data communication with other devices, an input interface function for accepting various instructions from the patient P, and a display function for displaying various types of information.

### [Processing Flow]

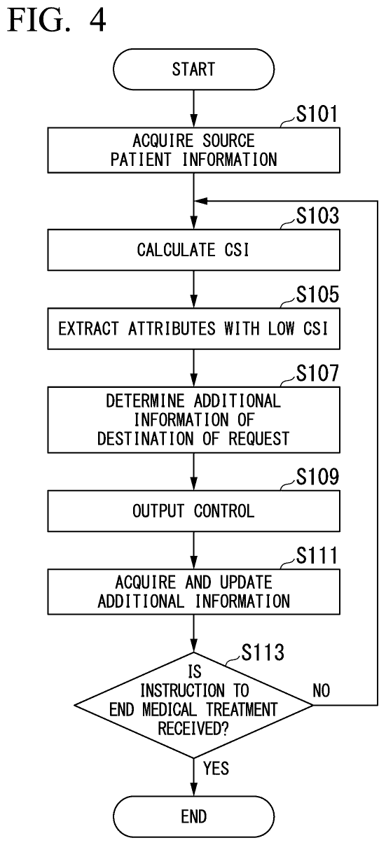

Next, a flow of medical support processing in the diagnosis support apparatus 1 will be described. FIG. 4 is a flowchart showing an example of medical support processing of the diagnosis support apparatus 1 according to the first embodiment. The medical support processing shown in FIG. 4 begins, for example, when the doctor D, who is treating the patient P, issues an instruction to start the medical support processing via the input interface of the doctor terminal device T1. At this time, the patient P and the doctor D may be in the same space, such as a hospital examination room (face-to-face medical treatment), or in separate spaces (online medical treatment).

First, the acquisition function 11 acquires source patient information, which is the source of patient information, from the medical database 3 via the communications network NW (step S101). The source patient information includes, for example, various types of patient test data (measurement values, images, etc.), electronic medical records, and response data to medical questionnaires. For example, the acquisition function 11 acquires the source patient information from the medical database 3 using, as a key, a patient identifier (patient ID) that identifies the patient and is input by the doctor D using the doctor terminal device T1. The acquisition function 11 registers the acquired source patient information in the patient information PI for each attribute. If past patient information on the patient P is stored in the memory 30, the acquisition function 11 also acquires this past patient information.

Next, the calculation function 12 calculates CSI for the source patient information acquired by the acquisition function 11 (step S103). For example, the calculation function 12 calculates the CSI from the perspective of the 5Ws (who, what, where, when, and why) and registers the same in the patient information PI.

Next, the extraction function 13 extracts a predetermined number of attributes (patient information) with low CSI when the attributes included in the patient information PI have been sorted in order of size of CSI (step S105). For example, the extraction function 13 sorts attributes determined to have “low CSI” in order of size of CSI, and extracts a predetermined number of attributes (e.g., the three worst) in ascending order of CSI.

Next, the determination function 14 determines, for the attributes extracted by the extraction function 13, additional information that is desired to be acquired in addition to the information acquired by the acquisition function 11, and a destination of request for this additional information (step S107).

The output control function 15 outputs request information for requesting provision of additional information from the destination of request (step S109). For example, the output control function 15 outputs information for requesting provision of the additional information from the destination of request (such as the doctor and/or the patient) to a terminal device (such as the doctor terminal device T1 and/or the patient terminal device T2). As a result, the information (hereinafter referred to as “request information”) for requesting provision of the additional information is displayed on the doctor terminal device T1 and/or patient terminal device T2.

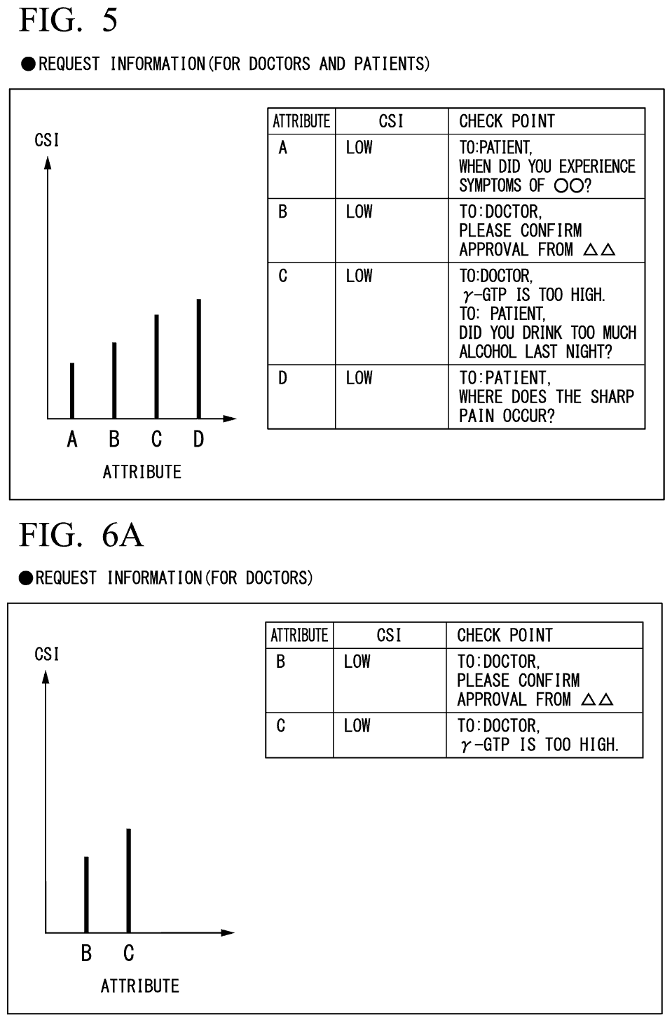

FIG. 5 shows an example of request information (for doctors and patients) according to the first embodiment. The request information in FIG. 5 displays information about four attributes (A, B, C, and D) with low CSI. For example, for attribute A, the request information displays a check point, “To: Patient, when did you experience symptoms of ◯◯?,” requesting provision of additional information from the patient that is a destination of request to improve the CSI from the “when” perspective. For attribute B, for example, the request information displays a check point, “To: Doctor, please confirm approval from ΔΔ,” requesting provision of additional information from the doctor that is a destination of request to improve the CSI from the “who” perspective. After checking this request information, the doctor and/or the patient can input responses to the check points by operating the doctor terminal device T1 and/or the patient terminal device T2.

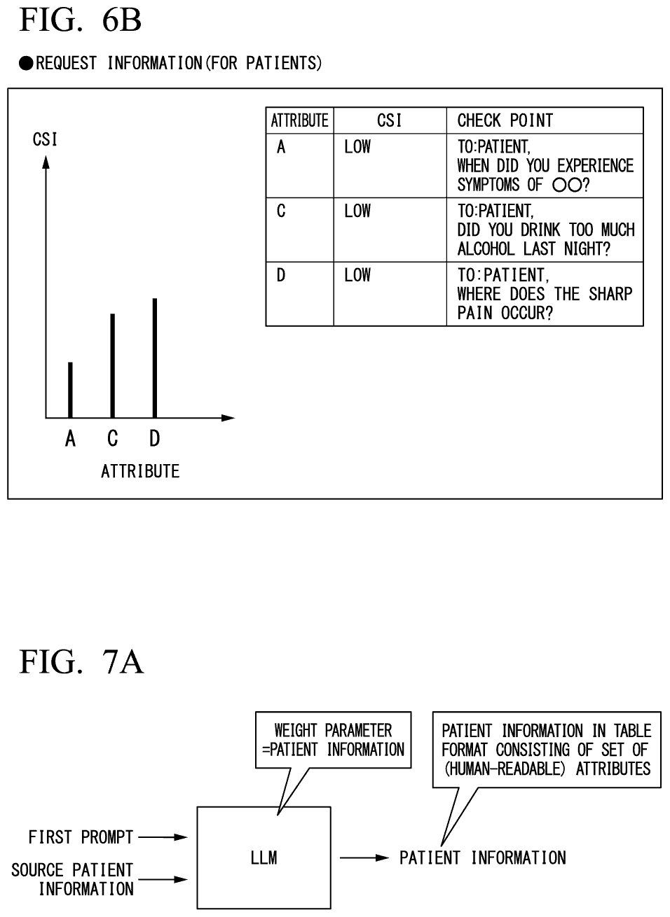

FIG. 6A shows an example of request information (for doctors) according to the first embodiment. The request information in FIG. 6A displays information about two attributes (B and C) among attributes with low CSI, for which a destination of request is a doctor, among attributes with low CSI. For example, for attribute C, the request information displays a check point, “To: Doctor, γ-GTP is too high,” requesting provision of additional information from the doctor that is the destination of request to improve the CSI from the “what” perspective. After checking this request information, the doctor can input a response to the check point by operating the doctor terminal device T1.

FIG. 6B shows an example of request information (for patients) according to the first embodiment. The request information in FIG. 6B displays information about three attributes (A, C, and D) among attributes with low CSI, for which a destination of request is a patient. For example, for attribute C, the request information displays a check point, “To: Patient, Did you drink too much alcohol last night?,” requesting provision of additional information from the patient that is the destination of request to improve the CSI from the “why” perspective. After checking this request information, the patient can input a response to the check point by operating the patient terminal device T2. Such request information for patients may be presented to the patient (patient terminal device T2) when the patient fills out a medical questionnaire conducted prior to medical treatment. Furthermore, compared to the request information for doctors, wording used for the request information for patients may be simpler and free of technical jargon.

Referring back to FIG. 4, next, the acquisition function 11 acquires additional information from the doctor terminal device T1 and/or the patient terminal device T2, and the management function 16 uses the acquired additional information to update the patient information PI stored in the memory 30 (step S111). Furthermore, the calculation function 12 recalculates CSI using the patient information to be updated (information regarding disease risk) and the additional information, and the management function 16 uses the recalculated CSI to update the patient information PI stored in the memory 30. Here, the management function 16 may add or delete attributes themselves in the patient information PI stored in the memory 30.

Next, the doctor D inputs an instruction to end medical treatment by operating the doctor terminal device T1, and if the acquisition function 11 accepts this instruction (step S113; YES), processing of this flowchart ends. On the other hand, if the acquisition function 11 does not receive the instruction to end the treatment (step S113; NO) (for example, if the acquisition function 11 receives an instruction to recalculate the CSI), the processing returns to processing of step S103 and repeats the subsequent steps. By repeating such processing, it is expected that attributes necessary for the treatment will remain and the CSI of each attribute will increase at the end of the treatment.

According to the first embodiment described above, it is possible to perform accurate and thorough continuous confirmation of patient information by both a subject and a medical professional. This enables systematic organization and visualization of patient information managed in a clinical decision support system (CDS). It is also possible to reduce the risk of misdiagnosis by the CDS. Furthermore, it is possible to provide input data with high reliability for other applications, such as various diagnosis support artificial intelligence (AI) model by using such patient information. In addition, when a subject and a medical professional are separated in time and space, such as in telemedicine, patient information can be handled more accurately.

### Second Embodiment

A second embodiment will be described below. In the following description, components and functions identical to those of the first embodiment are designated by the same reference numerals as those in the first embodiment, and detailed descriptions will be omitted. The second embodiment differs from the first embodiment in that patient information to be managed is a collection of parameters (weight parameters). The parameters are, for example, weight parameters of large language models (LLMs). That is, patient information includes weight parameters of large language models set by learning patient information (information regarding disease risk) and CSI.

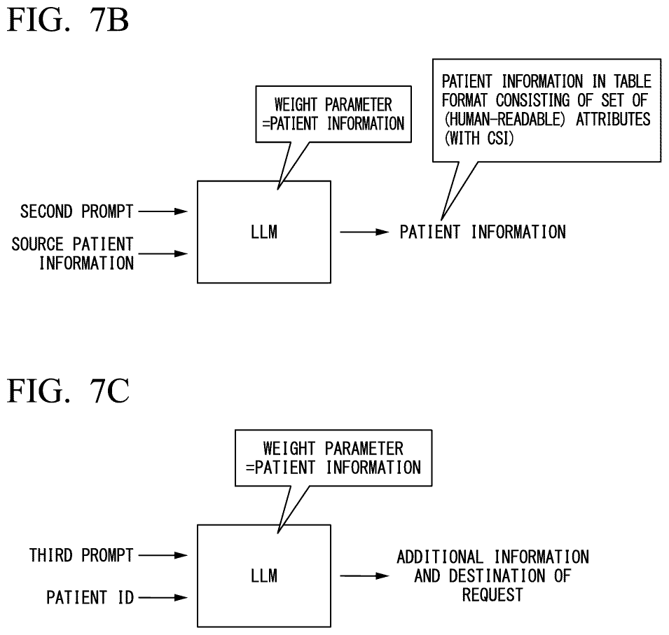

The management function 16 according to the second embodiment inputs source patient information acquired by the acquisition function 11 to an LLM. The LLM then manages patient information (using weight parameters thereof). FIG. 7A is a diagram showing an example of input/output of the LLM according to the second embodiment. The management function 16 inputs the source patient information acquired by the acquisition function 11 and a first prompt for outputting the patient information included in the LLM to the LLM. As a result, the LLM outputs the patient information included in the LLM in a table format consisting of a set of attributes, such as that shown in FIG. 2 of the first embodiment, such that people easily view the patient information.

FIG. 7B is a diagram showing another example of input/output of the LLM according to the second embodiment. The calculation function 12 inputs the source patient information acquired by the acquisition function 11 and a second prompt for calculating CSI for the source patient information to the LLM. This second prompt includes an instruction to calculate CSI using calculation logic (5 W) similar to that of the first embodiment. As a result, the LLM outputs the patient information (with CSI) included in the LLM in a table format consisting of a set of attributes, such as that shown in FIG. 2 of the first embodiment, such that people easily view the patient information.

FIG. 7C is a diagram showing another example of input/output of the LLM according to the second embodiment. The extraction function 13 and the determination function 14 input a patient ID and a third prompt for calculating additional information and a destination of request to the LLM. This third prompt includes instructions for executing functions similar to those of the extraction function 13 and the determination function 14 of the first embodiment. As a result, the LLM outputs the additional information and the destination of request (for example, information indicating a format for accepting and answering questions from the destination of request).

The input and output of the LLM described above are merely examples, and various configurations are possible. For example, the first to third prompts, which are examples of input data, may be integrated into one.

According to the second embodiment described above, it is possible to perform accurate and thorough continuous confirmation of patient information by both a subject and a medical professional. Furthermore, subject information with various formats and characteristics can be comprehensively and simply managed by managing weight parameters as subject information (patient information). Furthermore, subject information can be provided in a desired format depending on the application by rewriting prompts input into an LLM, thereby expanding the use of the subject information.

### Third Embodiment

A third embodiment will be described below. In the following description, components and functions identical to those of the first embodiment will be designated by the same reference numerals as those in the first embodiment, and detailed description will be omitted. The third embodiment differs from the first embodiment in that patient information to be managed is a simulation model. That is, the patient information includes a simulation model generated on the basis of patient information (information on disease risk).

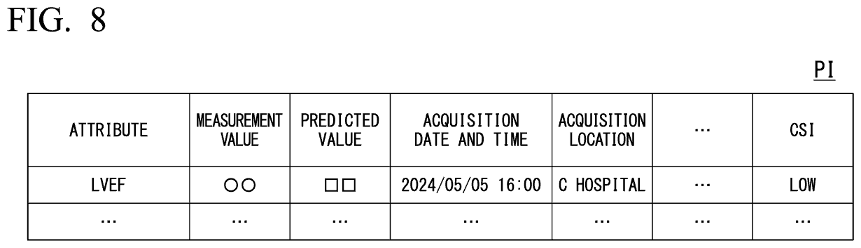

FIG. 8 is a diagram showing an example of patient information according to the third embodiment. FIG. 8 shows a case in which the patient information is a cardiac simulation model, and attributes to be managed include simulable cardiac data (e.g., left ventricular ejection fraction (LVEF)). When calculating CSI, the calculation function 12 performs calculations based on simulation results in addition to the same calculation logic (5 W) as in the first embodiment. The calculation function 12 compares actual data (e.g., measurement values for LVEF) with the simulation results of the simulation model (e.g., predicted values for LVEF), and if the simulation results (predicted values) deviate significantly from the actual data (measurement values) or if the simulation results cannot be calculated at all, calculates a low CSI for that data. In this case, the determination function 14 determines “confirmation of measurement values (request for re-measurement)” or “approval request for updating the simulation model such that the output of the simulation model approaches the measurement values” as additional information for the doctor D that is a destination of request. In response, the output control function 15 outputs request information for requesting provision of additional information from the destination of request to the doctor terminal device T1.

According to the third embodiment described above, it is possible to perform accurate and thorough continuous confirmation of subject information by both a subject and a medical professional. Furthermore, by managing a simulation model as subject information (patient information), calculating CSI thereof, and providing suggestions for improvement, the accuracy of the simulation model can be improved.

Some or all of the configurations illustrated in the first to third embodiments above may be combined and implemented.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnosis support apparatus comprising processing circuitry configured to:
analyze a medical image of a subject captured by a medical imaging device to acquire information regarding a disease risk of the subject;
calculate a credibility of the acquired information regarding the disease risk;
determine additional image information desired to be acquired in addition to the acquired information regarding the disease risk and a destination of request for the additional image information based on the calculated credibility; and
output request information for requesting provision of the determined additional image information from the destination of request.

2. A diagnosis support apparatus comprising processing circuitry configured to:
acquire information regarding a disease risk of a subject;
calculate credibility of the acquired information regarding the disease risk;
determine additional information desired to be acquired in addition to the acquired information regarding the disease risk and a destination of request for the additional information based on the calculated credibility; and
output request information for requesting provision of the determined additional information from the destination of request.

3. The diagnosis support apparatus according to claim 2, wherein the processing circuitry is further configured to output, to a terminal device, information for requesting provision of the additional information from the destination of request.

4. The diagnosis support apparatus according to claim 2, wherein the processing circuitry is further configured to output, to a terminal device of a medical professional, information for requesting provision of the additional information from the destination of request that is the medical professional.

5. The diagnosis support apparatus according to claim 2, wherein the processing circuitry is further configured to output, to a terminal device of the subject, information for requesting provision of the additional information from the destination of request that is the subject.

6. The diagnosis support apparatus according to claim 3, wherein the information includes the information regarding the disease risk, the credibility, and a character string requesting provision of the additional information from the destination of request.

7. The diagnosis support apparatus according to claim 2, wherein the processing circuitry is further configured to store subject information based on the information regarding the disease risk and the credibility in a storage device.

8. The diagnosis support apparatus according to claim 7, wherein the processing circuitry is further configured to:
acquire the additional information provided by the destination of request in response to the request information; and
update the subject information stored in the storage device using the acquired additional information.

9. The diagnosis support apparatus according to claim 8, wherein the processing circuitry is further configured to:
recalculate the credibility using the information regarding the disease risk and the additional information; and
update the subject information stored in the storage device using the recalculated credibility.

10. The diagnosis support apparatus according to claim 7, wherein the processing circuitry is further configured to:
extract a predetermined number of pieces of subject information with low credibility when sorting the subject information in order of size of credibility; and
determine the additional information and the destination of request with respect to the extracted predetermined number of pieces of subject information.

11. The diagnosis support apparatus according to claim 7, wherein the subject information includes weight parameters of a large language model set by learning the information regarding the disease risk and the credibility.

12. The diagnosis support apparatus according to claim 7, wherein the subject information includes a simulation model generated based on the information regarding the disease risk.

13. A diagnosis support method, using a computer, comprising:
acquiring information regarding a disease risk of a subject;
calculating a credibility of the acquired information regarding the disease risk;
determining additional information desired to be acquired in addition to the acquired information regarding the disease risk and a destination of request for the additional information based on the calculated credibility; and
outputting request information for requesting provision of the determined additional information from the destination of request.

14. A non-transitory computer-readable storage medium storing a program causing a computer to:
acquire information regarding a disease risk of a subject;
calculate a credibility of the acquired information regarding the disease risk;
determine additional information desired to be acquired in addition to the acquired information regarding the disease risk and a destination of request for the additional information based on the calculated credibility; and
output request information for requesting provision of the determined additional information from the destination of request.
